(19) United States
(12) Patent Application Publication
(10) Pub. No.: US 2022/0310202 A1
(43) Pub. Date: Sep. 29, 2022

(54) UTILIZATION OF SPARCE CODEBOOK IN MULTIPLEXED FLUORESCENT IN-SITU HYBRIDIZATION IMAGING

(71) Applicant: Applied Materials, Inc., Santa Clara, CA (US)

(72) Inventors: Bongjun Son, San Jose, CA (US); Chloe Kim, San Jose, CA (US); Yun-Ching Chang, San Jose, CA (US); Debjit Ray, Santa Clara, CA (US)

(21) Appl. No.: 17/698,769

(22) Filed: Mar. 18, 2022

Related U.S. Application Data
(60) Provisional application No. 63/166,204, filed on Mar. 25, 2021.

(51) Int. Cl. G16B 25/10; G06V 10/75; G06V 10/98; G06V 20/69

(52) U.S. Cl. CPC G16B 25/10; G06V 10/751; G06V 10/98; G06V 20/698; G06V 2201/04

(57) ABSTRACT

A method of method of spatial transcriptomics includes receiving a plurality of images of a sample from an mFISH imaging system and generating a pixel word represented by a sequence of N intensity values. For each pixel, the pixel word is compared to a codebook and a closest matching code word of a plurality of code words is identified. Each code word is represented by a sequence of N bits. The plurality of code words include a plurality of gene-identifying code words and a plurality of negative control code words, the plurality of negative control code words have an equal number of on-values, and on-values of the plurality of negative control code words are evenly distributed across the N bits such that each ordinal position in the sequence of N bits has a same total number of on-bits from the plurality of negative control code words.

### CROSS-REFERENCE TO RELATED APPLICATIONS

This application claims the benefit of priority to U.S. application Ser. No. 63/166,204, filed on Mar. 25, 2021, the contents of which are hereby incorporated by reference.

### TECHNICAL FIELD

This specification relates to sparse-code utilization for mFISH imaging.

### BACKGROUND

Multiplexed fluorescence in-situ hybridization (mFISH) imaging is a powerful technique to determine gene expression in spatial transcriptomics. In brief, a sample is exposed to multiple oligonucleotide probes that target RNA of interest. Then sequential rounds of fluorescence images are acquired with exposure to excitation light of different wavelengths and/or photobleaching followed by exposure to further rounds of oligonucleotide probes. For each given pixel, the fluorescence intensities from the different images form a signal sequence. This sequence is then compared to a library of reference codes from a codebook that associates each code with a gene. The best matching reference code is used to identify an associated gene that is expressed at that pixel in the image.

The codebook used to identify genes can include a number of negative control code words. These code words are generated by randomly assigning an on- or off-value to each bit of a code word, creating signal sequences that do not correspond to any gene in the sample. The negative control code words are used to differentiate true positive, false positive, and blank matches found in image sequences generated during imaging. The signal corresponding to the most commonly matched negative control code word determines the lowest signal that needs additional identification information to be confidently be matched to a gene.

### SUMMARY

In one aspect, a method of spatial transcriptomics includes receiving a plurality of images of a sample from an mFISH imaging system, for each pixel of a plurality of pixels registered across the plurality of images generating a pixel word from intensity values of each pixel of the plurality of pixels of the plurality of images with each pixel word represented by a sequence of N intensity values. For each pixel of the plurality of pixels, the pixel word for the pixel is compared to a codebook including a plurality of code words, and a closest matching code word of the plurality of code words to the pixel word is identified. Each code word is represented by a sequence of N bits. The plurality of code words include a plurality of gene-identifying code words and a plurality of negative control code words, and the plurality of negative control code words have an equal number of on-values. On-values of the plurality of negative control code words are evenly distributed across the N bits such that each ordinal position in the sequence of N bits has a same total number of on-bits from the plurality of negative control code words. A gene or error associated with the closest matching code word is determined, and for at least one pixel of the plurality of pixels an association of the pixel with the gene or error is stored.

In another aspect, a method of generating a codebook includes obtaining a first plurality of gene-identifying code words for the codebook. Each gene-identifying code word of the plurality of gene-identifying code words is represented by a sequence of N bits. Each code word of the first subset of code words includes a sequence of bits, and the sequence of bits correspond to a best match to a pixel data value identifying a gene. A plurality of negative control code words is generated, each negative control code word of the plurality of gene-identifying code words represented by a sequence of N bits. The plurality of negative control code words have an equal number of on-values. On-values of the plurality of negative control code words are evenly distributed across the N bits such that each ordinal position in the sequence of N bits has a same total number of on-bits from the plurality of negative control code words, and a Hamming distance between each negative control code word and each gene-identify code word is at least a distance threshold.

Advantages of implementations can include, but are not limited to, one or more of the following.

Disclosed herein is a method for generating a codebook for identifying gene targets during mFISH imaging where the negative control code words are generated with uniform numbers of on-values in each code word, and in each position across all code words. This method reduces possible degeneracy between negative control code word positions and ensures the set of code words achieves more uniform Hamming distance separation between codebook gene code words. The uniform distribution of on-off values in the set of negative control code words decreases the occurrence of false-positive matches thereby increasing the signal confidence of true-positive gene identifications and allowing more gene targets to be correctly identified without increasing the size of the codebook.

Increased positive gene identification per sequence of collected images leads to higher overall assay throughput as well as higher confidence in the results, reducing the collection of inconclusive data and increasing assay reproducibility. By filtering fewer false-positives and increasing confidence in collected signals, reagent usage is also reduced resulting in monetary benefit. Downstream analysis is also improved with regard to a lower false-positive rate and better filtration of sequence matches to negative controls.

The details of one or more embodiments are set forth in the accompanying drawings and the description below. Other features, aspects, and advantages will become apparent from the description, the drawings, and the claims.

### BRIEF DESCRIPTION OF THE DRAWINGS

FIG. 1 is a schematic diagram of an apparatus for multiplexed fluorescence in-situ hybridization imaging.

FIG. 2 is a flow chart of a method of data processing.

FIG. 3 illustrates a method of decoding.

FIG. 4A is a table of negative control code words for a codebook where the on-values are randomly placed.

FIG. 4B is a table of negative control code words for a codebook where the on-values are uniformly distributed across the columns of the code words.

FIG. 5A is a confidence-cutoff chart which uses randomly distributed negative control code words.

FIG. 5B is a confidence-cutoff chart which uses uniformly distributed negative control code words.

Like reference numbers and designations in the various drawings indicate like elements.

### DETAILED DESCRIPTION

Current approaches to generating the set of negative control code words in a codebook utilize random sequences of sparse binary code containing randomly-placed on-values, with a relatively low Hamming weight (e.g., number of on-values) compared to the length of each code word in the codebook (e.g., 25% or less of total code word length). A Hamming distance to the nearest gene-identifying code word of the codebook is calculated for each negative control code word, and negative control code words having a Hamming distance less than a distance threshold value are discarded and randomly generated again. Once all generated negative control code words exceed the Hamming distance threshold to the rest of the gene-identifying code words, the codebook is complete. This codebook is then used to deconvolute the multiplexed signals at each pixel location in a sequence of collected mFISH images and match the signal sequences to code words corresponding to gene targets for gene identification. The randomly-generated negative control code words serve as a filter for false-positives and matches to known negative control code words.

However, this approach of using randomly generated code words can lead to inconsistent bit position degeneracy, where every bit at a given column position within the set of negative control code words is either an on- or off-value across all negative control code words (e.g., all on- or all off-values in a bit position). This leads to inconsistent signal normalization and necessary assay iterations to increase data confidence. These issues are key issues causing decreased analysis throughput. Inefficiencies in identifying false-positives and high negative control signal results in duplication of work product, leading to inconsistent data output, increased reagent use, and reduced assay throughput.

An advantageous approach to creating the set of negative control code words includes a two-step process: creating code words in which each code word contains a known number of on-value bits (e.g., 1s); and creating code words with a uniform distribution of on-bits across all column positions. This method maintains the same Hamming distance threshold and increases overall quality of collected data leading to increases in assay throughput, reduction in reagents used, and reduced project times.

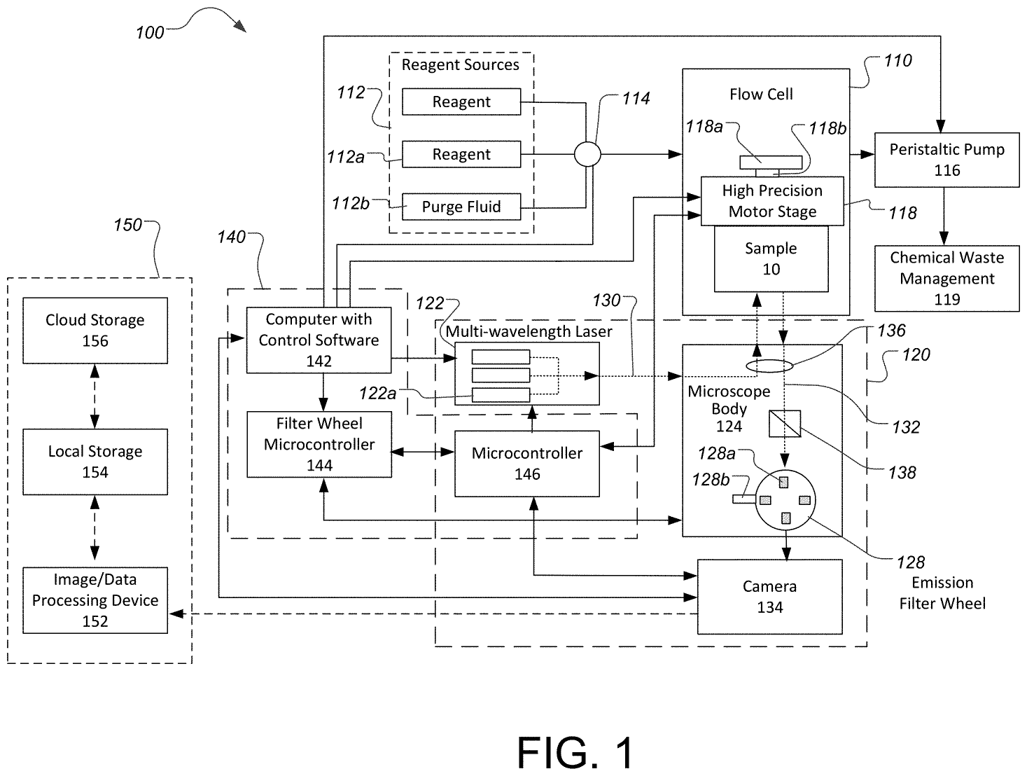

Referring to FIG. 1, a multiplexed fluorescent in-situ hybridization (mFISH) imaging and image processing apparatus 100 includes a flow cell 110 to hold a sample 10, a fluorescence microscope 120 to obtain images of the sample 10, and a control system 140 to control operation of the various components of the mFISH imaging and image processing apparatus 100. The control system 140 can include a computer 142, e.g., having a memory, processor, etc., that executes control software.

The fluorescence microscope 120 includes an excitation light source 122 that can generate excitation light 130 of multiple different wavelengths. In particular, the excitation light source 122 can generate narrow-bandwidth light beams having different wavelengths at different times. For example, the excitation light source 122 can be provided by a multi-wavelength continuous wave laser system, e.g., multiple laser modules 122a that can be independently activated to generate laser beams of different wavelengths. Output from the laser modules 122a can be multiplexed into a common light beam path.

The fluorescence microscope 120 includes a microscope body 124 that includes the various optical components to direct the excitation light from the light source 122 to the flow cell 110. For example, excitation light from the light source 122 can be coupled into a multimode fiber, refocused and expanded by a set of lenses, then directed into the sample by a core imaging component, such as a high numerical aperture (NA) objective lens 136. When the excitation channel needs to be switched, one of the multiple laser modules 122a can be deactivated and another laser module 122a can be activated, with synchronization among the devices accomplished by one or more microcontrollers 144, 146.

The objective lens 136, or the entire microscope body 124, can be installed on vertically movable mount coupled to a Z-drive actuator. Adjustment of the Z-position, e.g., by a microcontroller 146 controlling the Z-drive actuator, can enable fine tuning of focal position. Alternatively, or in addition, the flow cell 110 (or a stage 118 supporting the sample in the flow cell 110) could be vertically movable by a Z-drive actuator 118b, e.g., an axial piezo stage. Such a piezo stage can permit precise and swift multi-plane image acquisition.

The sample 10 to be imaged is positioned in the flow cell 110. The flow cell 110 can be a chamber with cross-sectional area (parallel to the object or image plane of the microscope) with an area of about 2 cm by 2 cm. The sample 10 can be supported on a stage 118 within the flow cell, and the stage 118 (or the entire flow cell 110) can be laterally movable, e.g., by a pair of linear actuators 118a to permit XY motion. This permits acquisition of images of the sample 10 in different laterally offset fields of view (FOVs). Alternatively, the microscope body 124 could be carried on a laterally movable stage.

An entrance to the flow cell 110 is connected to a set of hybridization reagents sources 112. A multi-valve positioner 114 can be controlled by the controller 140 to switch between sources to select which reagent 112a is supplied to the flow cell 110. Each reagent includes a different set of one or more oligonucleotide probes. Each probe targets a different RNA sequence of interest, and has a different set of one or more fluorescent materials, e.g., phosphors, that are excited by different combinations of wavelengths. In addition to the reagents 112a, there can be a source of a purge fluid 112b, e.g., deionized (DI) water.

An exit to the flow cell 110 is connected to a pump 116, e.g., a peristaltic pump, which is also controlled by the controller 140 to control flow of liquid, e.g., the reagent or purge fluid, through the flow cell 110. Used solution from the flow cell 110 can be passed by the pump 116 to a chemical waste management subsystem 119.

In operation, the controller 140 causes the light source 122 to emit the excitation light 130, which causes fluorescence of fluorescent material in the sample 10, e.g., fluorescence of the probes that are bound to RNA in the sample and that are excited by the wavelength of the excitation light. The emitted fluorescent light 132, as well as back propagating excitation light, e.g., excitation light scattered from the sample, stage, etc., are collected by an objective lens 136 of the microscope body 124.

The collected light can be filtered by a multi-band dichroic mirror 138 in the microscope body 124 to separate the emitted fluorescent light from the back propagating illumination light, and the emitted fluorescent light is passed to a camera 134. The camera 134 can be a high resolution (e.g., 2048×2048 pixel) CMOS (e.g., a scientific CMOS) camera, and can be installed at the immediate image plane of the objective. When triggered by a signal, e.g., from a microcontroller, image data from the camera can be captured, e.g., sent to an image processing system 150. Thus, the camera 134 can collect a sequence of images from the sample.

To further remove residual excitation light and minimize cross talk between excitation channels, each laser emission wavelength can be paired with a corresponding band-pass emission filter 128a. Each filter 128a can have a wavelength of 10-50 nm, e.g., 14-32 nm. The filters are installed on a high-speed filter wheel 128 that is rotatable by an actuator 128b. The filter wheel 128 can be installed, e.g., at the infinity space, to minimize optical aberration in the imaging path. After passing the emission filter of the filter wheel 128, the cleaned fluorescence signals can be refocused by a tube lens and captured by the camera 134. The dichroic mirror 138 can be positioned in the light path between the objective lens 138 and the filter wheel 128.

The control software coordinates communication between the computer 142 and the device components of the apparatus 100. This control software can integrate drivers of all the device components into a single framework, and thus can allow a user to operate the imaging system as a single instrument (instead of having to separately control many devices).

Fluorescence images are acquired for each combination of possible values for the z-axis, color channel (excitation wavelength), lateral FOV, and reagent. A data processing system 150 is used to process the images and determine gene expression to generate the spatial transcriptomic data. At a minimum, the data processing system 150 includes a data processing device 152, e.g., one or more processors controlled by software stored on a computer readable medium, and a local storage device 154, e.g., non-volatile computer readable media, that receives the images acquired by the camera 134.

In some implementations, the data processing system 150 performs on-the-fly image processing as the images are received. In particular, while data acquisition is in progress, the data processing device 152 can perform image pre-processing steps, such as filtering and deconvolution, that can be performed on the image data in the storage device 154 but which do not require the entire data set.

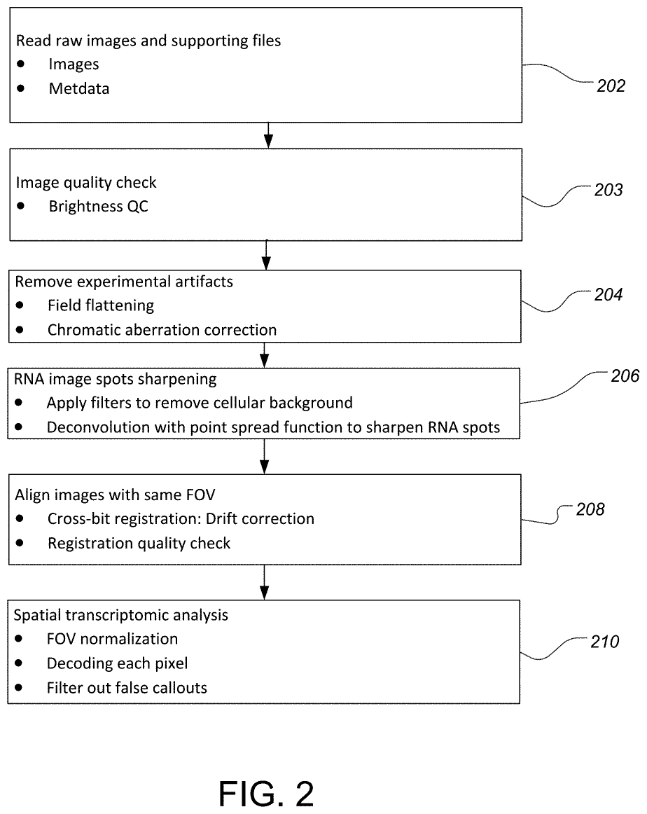

FIG. 2 illustrates a flow chart of a method of data processing in which the processing is performed after all of the images have been acquired. The process begins with the system receiving the raw image files and supporting files, e.g., metadata (step 202). In particular, the data processing system can receive the full set of raw images from the camera, e.g., an image for each combination of possible values for the z-axis, color channel (excitation wavelength), lateral FOV, and reagent.

The image files received from the camera can optionally include metadata, the hardware parameter values (such as stage positions, pixel sizes, excitation channels, etc.) at which the image was taken. The data schema provides a rule for ordering the images based on the hardware parameters so that the images are placed into one or more image stacks in the appropriate order. If metadata is not included, the data schema can associate an order of the images with the values for the z-axis, color channel, lateral FOV and reagent used to generate that image.

The collected images can be subjected to one or more quality metrics (step 203) before more intensive processing in order to screen out images of insufficient quality. Only images that meet the quality metric(s) are passed on for further processing.

In order to detect regions of interest, a brightness quality value can be determined for each collected image. The brightness quality can be used to determine whether any cells are present in the image. For example, the intensity values of all the pixels in the image can be summed and compared to a threshold. If the total is less than the threshold, then this can indicate that there is essentially nothing in the image, i.e., no cells are in the image, and there is no information of interest and the image need not be processed.

Next, each image is processed to remove experimental artifacts (step 204). Since each RNA molecule will be hybridized multiple times with probes at different excitation channels, strict alignment across the multi-channel, multi-round image stack is beneficial for revealing RNA identities over the whole FOV. Removing the experimental artifacts can include field flattening and/or chromatic aberration correction.

Each image is processed to provide RNA image spot sharpening (step 206). RNA image spot sharpening can include applying filters to remove cellular background and/or deconvolution with point spread function to sharpen RNA spots. In order to distinguish RNA spots from a relatively bright background, a low-pass filter is applied to the image, e.g., to the field-flattened and chromatically corrected images to remove cellular background around RNA spots. The filtered images are further de-convolved with a 2-D point spread function (PSF) to sharpen the RNA spots, and convolved with a 2-D Gaussian kernel with half pixel width to slightly smooth the spots.

The images having the same FOV are registered to align the features, e.g., the cells or cell organelles, therein (step 208). To accurately identify RNA species in the image sequences, features in different rounds of images are aligned, e.g., to sub-pixel precision. In particular, high intensity regions should generally be located at the same position across multiple images of the same FOV. Techniques that can be used for registration between images include phase-correlation algorithms and mutual-information (MI) algorithms.

After registration of the images in a FOV, spatial transcriptomic analysis can be performed (step 210). First, intensity values in the image are normalized relative to the maximum intensity value in the image. For example, the maximum intensity value is determined, and all intensity values are divided by the maximum so that intensity values vary between 0 and I_{MAX}, e.g., 1.

Next the intensity values in the image are analyzed to determine an upper quantile that includes the highest intensity values, for example, the 99% and higher quantile (i.e., upper 1%). The intensity value at this quantile limit can be determined and stored. All pixels having intensity values within the upper quantile are reset to have the maximum intensity value, e.g., 1. Then the intensity values of the remaining pixels are binned and scaled to run to the same maximum (e.g., 1). To accomplish this, intensity values for the pixels that were not in the upper quantile are divided by the stored intensity value for the quantile limit.

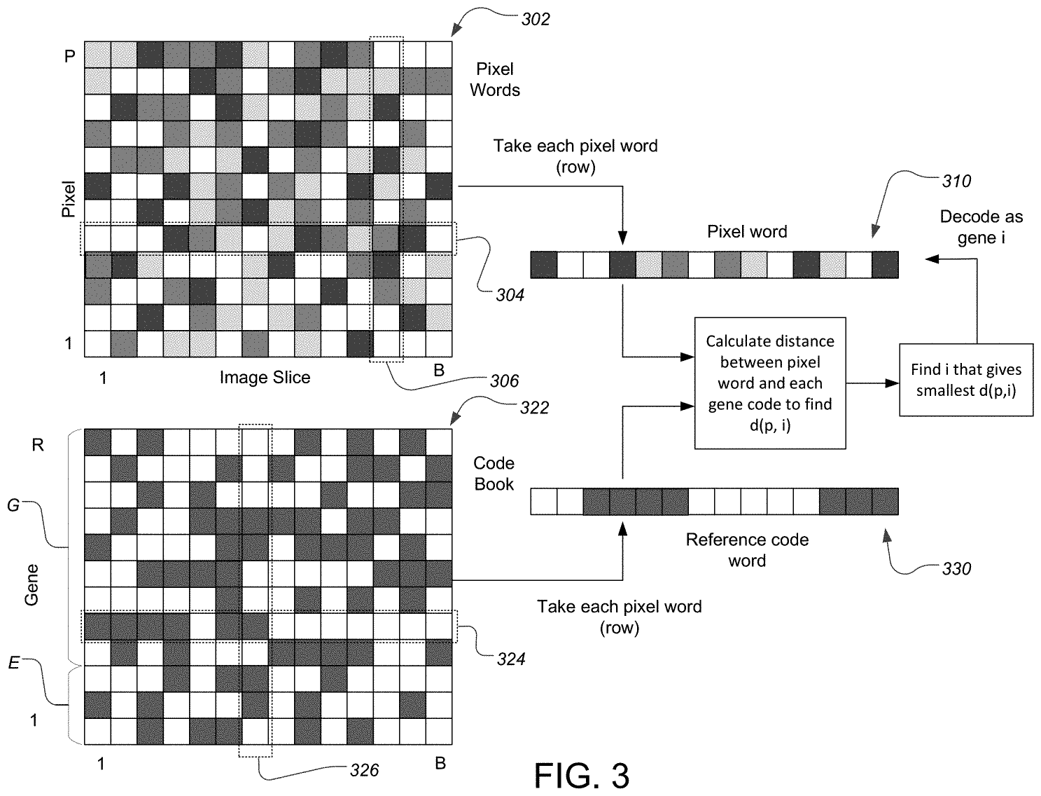

Decoding an image is explained with reference to FIG. 3. The aligned images for a particular FOV can be considered as a stack that includes multiple image layers, with each image layer being X by Y pixels, e.g., 2048×2048 pixels. The number of image layers, B, depends on the combination of the number of color channels (e.g., number of excitation wavelengths, N_channels) and number of hybridizations (e.g., number of reactants, N_hybridizations), e.g., B=N_hybridization*N_channels. In some implementations, B=16.

After normalization, this image stack is evaluated as a 2-D matrix 302 of pixel words. The matrix 302 has P rows 304, where P=X*Y, and B columns 306, where B is the number of images in the stack for a given FOV. Each row 304 corresponds to one of the pixels (the same pixel across the multiple images in the stack), the intensity values from the row 304 represent a pixel word 310. Each column 306 provides one of the values in the word 310, i.e., the intensity value from the image layer for that pixel. As noted above, the values can be normalized, e.g., vary between 0 and I_{MAX}. Different intensity values are represented in FIG. 3 as different degrees of shading of the respective cells.

The data processing system 150 stores a codebook 322 that is used to decode the image data to identify the gene expressed at the particular pixel. The codebook 322 includes multiple reference code words, and each reference code word is associated with either a particular gene or a negative control code word. As shown in FIG. 3, the codebook 322 can be represented as a 2D matrix with R rows 324, and B columns 326. The R rows 324 include a first group of G rows, where G is the number of gene-identifying code words, e.g., the number of genes the codebook 322 can decode, and a second group of E rows, where E is the number of negative control code words. Typically R=G+E. The codebook 322 of FIG. 3 includes 12 columns (e.g., B=12), and in some embodiments, B can be more (e.g., B=16). The gene words, G, are established by prior calibration and correspond to the expected pixel word of known genes. The design of negative control words, E, is described further below.

Each row 324 contains a sequence of B values (e.g., bits) and corresponds to one of the code words 330, either a gene-identifying code word or a negative control code word, and each column 326 provides one of the values in the reference code word 330. For each column 326, the values in the reference code 330 can be binary, i.e., “on” or “off” For example, each value can be either 0 or I_{MAX}, e.g., 1. The on and off values are represented in FIG. 3 by light and dark shading of respective cells.

Each code word of B values has 2^{B} assignable combinations of values. However, utilizing a portion of these total assignable values for gene- or negative control words and leaving the remaining portion unassigned allows for a negative control design of the codebook 322. The codebook 322 maintains two parameters across all rows 324: each row 324 shares the same Hamming weight (H_{W}) and minimum Hamming distance (H_{D}) from other rows 324.

The H_{W} of a code word is the number of on-values per row 324 and a uniform H_{W} between rows 324 reduces disproportionate pixel value misidentification bias. Additionally, maintaining a low H_{W} (e.g., four on-values per row) in the rows 324 compared to the total code word length of the codebook 322 further reduces misidentification frequency, thereby increasing accuracy.

The H_{D} between each row 324 is the number of positions at which two numerical strings of equal length, e.g., a reference string and a code string, are different and is calculated as a sum of absolute differences between each value position in a code string and corresponding reference string, a means of measuring the information-distance between two binary strings. In other words, it measures the minimum number of substitutions required to change one string into the other, or the minimum number of errors that could transform one string into the other. For example, given two six digit strings,
Reference: 010101
Code: 011001
   the H_{D} would be 2, the strings requiring two value substitutions (e.g., at the third and fourth position) to interconvert. This calculation can be expressed as:

$${H}_{D} = {\sum }_{i = 1}^{B} \vert {x}_{i} - {y}_{i} \vert$$

where H_{D}, using the codebook 322 as a reference, has inclusive limits between 0 (e.g., identical strings) and B, the total number of columns (e.g., orthogonal strings). The information-distance criteria used to design the negative control code words can be a minimum, maximum, or exact value Hamming distance between the words of the codebook. If code words separated by a Hamming distance of 2 or more are used, then no single value-error (e.g., a “0” misidentified as a “1”) can transform one code word into another, reducing the misidentification rate. Increasing the Hamming distance separation requirement further decreases the misidentification rate. In some implementations, the Hamming distance can be at least four (e.g., >4).

The codebook 322 includes a number of code words corresponding to negative control words that when matched identify false-positive or known negative pixel words 310, non-sense words that do not correspond to any gene in the codebook 322. The negative control words are a number of rows 324 (E) that constitute a portion of the codebook 322 which includes between 5% to 25% of the total rows, R, of the codebook 322. For example, a codebook 322 including 140 rows 324 can reserve 132 rows corresponding to gene-identifying code words 330 (G) and 8 rows (˜6% of R) corresponding to negative control words (E). Using the example of FIG. 3, the codebook 322 includes 9 gene-identifying code words 330 (e.g. 75% of the total rows, R) and 3 negative control words (e.g., 25% of the total rows, R).

The codebook 322 can be generated algorithmically through the use of a coding language. The following example provides a method to generate a 140 word codebook 322 (e.g., codebook 322 in which R=140 and B=16, e.g., Xᵢⱼ where i=1, 2, . . . , 140, and j=1, 2, . . . , 16) including a set of M negative control code words (e.g., M=E<R and B=16, e.g., Yᵢⱼ where i=1, 2, . . . , M, and j=1, 2, . . . , 16).

The H_{W}, H_{D}, and the number of on-values (N) per bit position (e.g., column) of the negative control code words are defined for the codebook 322. In one example, the H_{W}=4, H_{D}=4, and the number of on-values is 2 (N=2, where N=iYᵢⱼ₍₂₎). To determine a set of the negative control code words (e.g., Mᵢⱼ that satisfies the above conditions, define a target array Lⱼ (j=1, 2, . . . , 16)=N. Subtract the first code word (i=1) from target array (Lⱼ−X₁ⱼ) and calculate an updated residue (S) such that S=Lⱼ−X₁ⱼ.

Add the first negative control code word (X₁ⱼ) to the set of M negative control code words (M₁ⱼ₍₂₎=X₁ⱼ). Determine the next negative control code word in remaining codebook (Xᵢⱼ where i≠1) by subtracting each remaining negative control code word from the residue calculated above such that S′=Lⱼ−Xᵢⱼ where i≠1 and {x∈Z|x=−1, 0, 1, . . . } (Z represents integer set).

Determine a second negative control code word (Xᵢ) that returns the lowest residue value and add the second negative control code word to the set of negative control code words (e.g., M₂ⱼ₍₁₎=X₂ⱼ).

Repeat the above steps until iMᵢⱼ₍₂₎=1. Update the codebook 322 X′={x|x∈X and x∈yᵢⱼ₁}. Find the n+1 subset Mᵢⱼ₍ₙ₋₁₎ by using the updated codebook 322 X′ and iterating the above steps until n+1=N. The final set of negative control code words (M) will be Mᵢⱼ=n=iMᵢⱼ₍ₙ₊₁₎.

Bit-switching errors occur at a low rate (>10%) and negative control words in a codebook 322 allow for increased confidence in identification of gene words through identification of sense- or non-sense pixel words including one or more errors. For example, if a value in a pixel word is incorrectly identified, e.g., a “0” identified as a “1”, or vice versa, the pixel word may no longer be within an information-distance of the correct gene word and thus be misidentified. This can lead to missed gene counts and if the corresponding gene word is too close in information-distance to a neighboring gene word, the pixel word may be misidentified as a second, incorrect gene word. Negative control words are designed with a number of criteria to create a minimum information-distance between each negative control word and distribute the values within each negative control word 330 uniformly across the columns 326 of the negative control rows E.

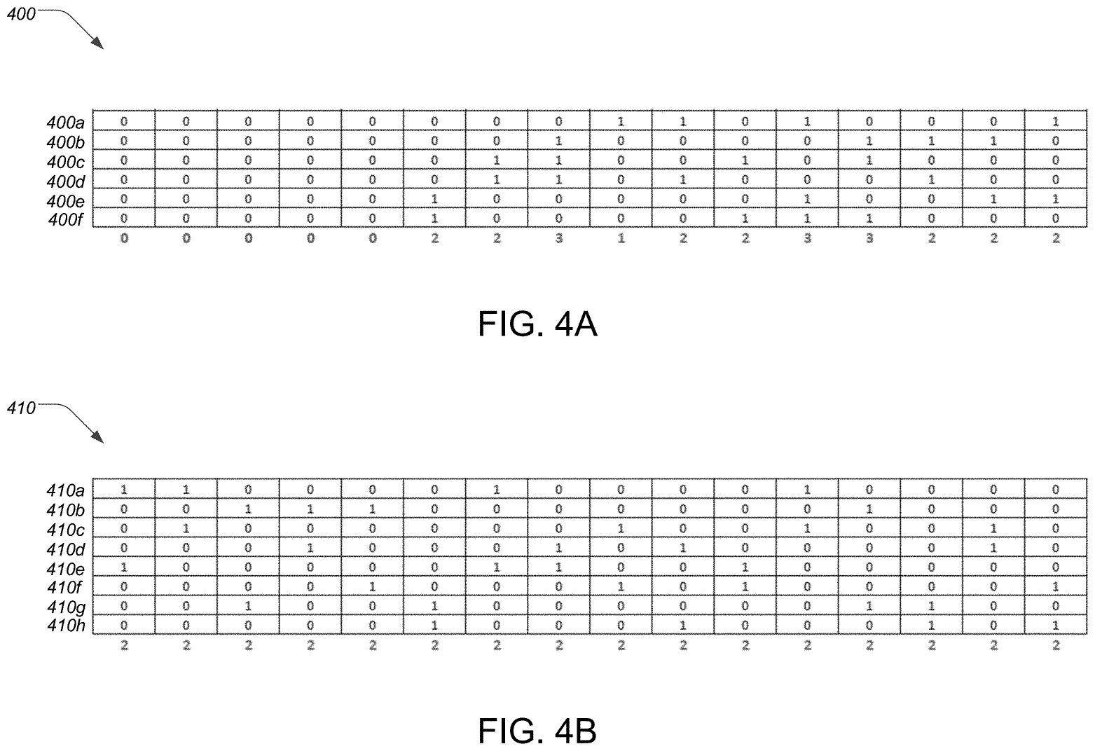

The technique described below for generating the negative control code words of the codebook 322 can provide additional layers of data integrity protection by creating symmetric information-distance between negative control and code word code words. The technique can also uniformly (e.g., evenly) distribute the number and arrangement of on-values across all columns of the codebook 322. FIGS. 4A and 4B are two example sets of negative control code words for codebook 322, illustrated in table form. Both example sets maintain the same constant H_{W} (e.g., 4), minimum H_{D} (e.g., >4) between code words (e.g., rows), and total number of columns (B=16) corresponding to each ordinal position of the 16-bit negative control code words. However, in FIG. 4A the ordinal positions of the on-bits were generated randomly, whereas in FIG. 4B the ordinal positions of the on-bits were generated to maintain a constant column sum value, e.g., same total number of on-bits. Beneath the tables of FIGS. 4A and 4B, each column is summed and a value representing the total number of on-bits is shown beneath the respective column (e.g., column 1 table 400 has a value of 0, whereas column 8 table 400 has a value of 3).

FIG. 4A depicts six negative control words 400a-f with 16 columns (B) in a table 400. The negative control words 400a-f were generated using a random value distribution. Randomly generated code word tables can include problematic arrangements, such as table 400, in which the first five value columns were determined to be “off” values (0). This results columns of the code word table 400 containing on-values carrying unequal weight in the distance calculation from gene words 704. Moreover, a zero column sum value in a large number of columns results in ordinal position on-value degeneracy, essentially creating a table of reduced bit-length and reducing the information-space available for distance calculations from 16 bits to 11, thereby decreasing the resolution of the negative control code words to identify false-positives.

FIG. 4B depicts eight negative control words 410a-h with 16 columns (B) in a table 410. The negative control words 400a-h were generated using a uniform column sum value distribution (e.g., evenly distributed), e.g., a constant sum value (e.g., 2) in all value columns. This additional negative control code word generation criteria ensures that all ordinal positions in the negative control words 410a-h have the same weight in the information-distance calculation when decoding pixels.

Referring again to FIG. 3, for each pixel to be decoded, a distance d(p,i) is calculated between the pixel word 310 and each reference code word 330. For example, the distance between the pixel word 310 and reference code word 330 can be calculated as a Euclidean distance, e.g., a sum of squared differences between each value in the pixel word and the corresponding value in the reference code word. This calculation can be expressed as:

$$d ( p , i ) = {\sum }_{x = 1}^{B} {( {I}_{p , x} - {C}_{i , x} )}^{2}$$

where I_{p,x} are the values from the matrix 302 of pixel words and C_{i,x} are the values from the matrix 322 of reference code words. Other metrics, e.g., sum of absolute value of differences, cosine angle, correlation, etc., can be used instead of a Euclidean distance.

Once the distance values for each code word are calculated for a given pixel, the smallest distance value is determined, the code word that provides that smallest distance value is selected as the closest matching code word. The gene corresponding to that closest matching code word is determined, e.g., from a lookup table that associates code words with genes, and the pixel is tagged as expressing the gene.

Returning to FIG. 2, the data processing apparatus can filter out false callouts. One technique to filter out false callouts is to discard tags where the distance value d(p,i) that indicated expression of a gene is greater than a threshold value, e.g., if d(p,i)>D_{IMAX}.

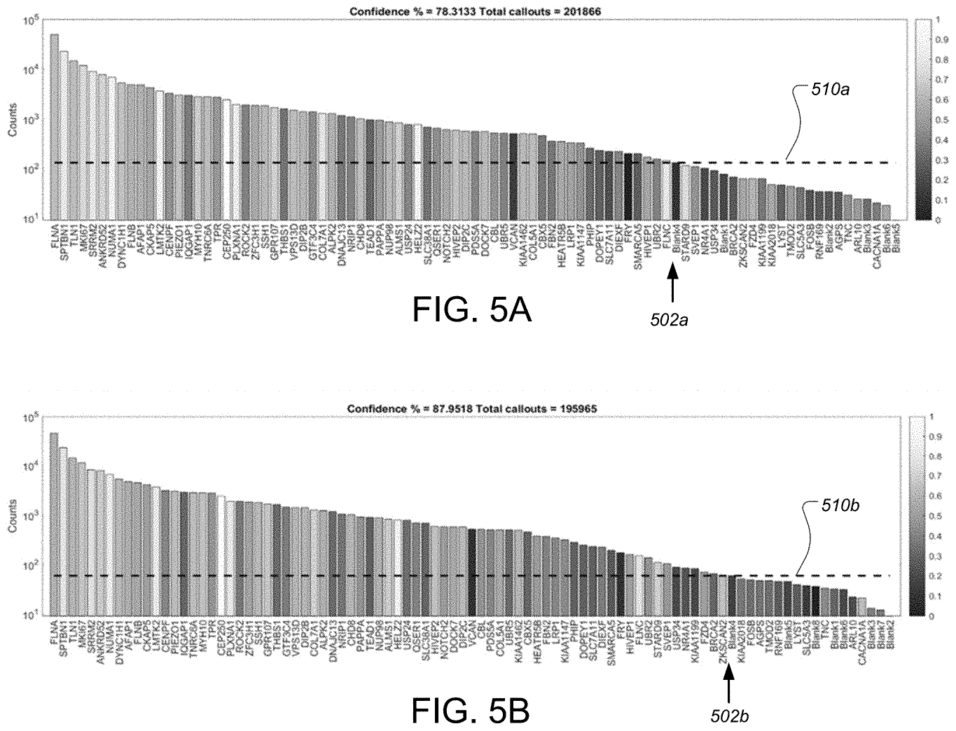

When the image stacking and gene word identification is complete, the maximum intensity values (e.g., counts) associated with a blank code word in the negative control words establishes a certainty threshold for filtering positive- from uncertain gene identifications. Gene code words below the certainty threshold can be raised above the certainty threshold with additional identification information. For example, FIG. 5A depicts a logarithmic histogram chart (e.g., counts versus code words) for a codebook of 83 gene words and 6 negative control words, after imaging, pixel decoding, and identification. FIG. 5A further includes a grey-scale to the right of the chart specifying the normalized confidence level of each individual code word, ranging from 1 (e.g., 100% confidence) to 0 (e.g., 0% confidence). The negative control words are labeled Blank1 through Blank6; gene words have other labels, e.g., FLNA, SPTBN1, etc. The negative control words of the codebook 322 for FIG. 5A were created using randomly distributed values across the columns of the codebook 322, the same process used to generate table 400 in FIG. 4A. The negative control word with the highest associated intensity value (e.g., “Blank4”, 502a) establishes the confidence threshold 510a for positive gene identification. Gene words are considered uncertain and not positively identified if their associated intensity value lies below threshold 510a, and confidently identified above threshold 510a.

At the top of FIG. 5A is the total ratio of positively identified genes to uncertain gene matches (e.g., Confidence). 65 of the total 83 gene words had an intensity value above the confidence threshold 510a for a ratio of 78.3%.

FIG. 5B depicts a histogram chart of the logarithmic intensity values (e.g., counts) for a codebook of 83 gene words and 8 negative control words. The negative control words for FIG. 5B were created using a uniform distribution of values across the columns of the codebook 322, the same process used to generate table 410 in FIG. 4B. The uniform distribution ensures an equal weight to every column of the table 410 and higher confidence in the identification of pixel words with both gene- and negative control words, as described above. Blank4 is the negative control word with the highest associated intensity value creating threshold 510b.

At the top of FIG. 5B, the total confidence ratio is 73 of the total 83 gene words had an intensity value above the confidence threshold 510a for a ratio of 87.9%, an improvement of 8 confidently identified gene words from the same data set as FIG. 5A.

Although the description above focuses on code words having 16 bits, the technique described is adaptable to code words of other bit lengths.

Embodiments of the subject matter described in this specification can be implemented in a computing system that includes a back-end component, e.g., as a data server, or that includes a middleware component, e.g., an application server, or that includes a front-end component, e.g., a client computer having a graphical user interface, a web browser, or an app through which a user can interact with an implementation of the subject matter described in this specification, or any combination of one or more such back-end, middleware, or front-end components. The components of the system can be interconnected by any form or medium of digital data communication, e.g., a communication network. Examples of communication networks include a local area network (LAN) and a wide area network (WAN), e.g., the Internet.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially be claimed as such, one or more features from a claimed combination can in some cases be excised from the combination, and the claimed combination may be directed to a subcombination or variation of a subcombination.

Similarly, while operations are depicted in the drawings and recited in the claims in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method of spatial transcriptomics, comprising:
receiving a plurality of images of a sample from an mFISH imaging system;
for each pixel of a plurality of pixels registered across the plurality of images, generating a pixel word from intensity values of each pixel of the plurality of pixels of the plurality of images, each pixel word represented by a sequence of N intensity values; and
for each pixel of the plurality of pixels,
comparing the pixel word for the pixel to a codebook including a plurality of code words and identifying a closest matching code word of the plurality of code words to the pixel word, each code word represented by a sequence of N bits, wherein the plurality of code words include a plurality of gene-identifying code words and a plurality of negative control code words, wherein the plurality of negative control code words have an equal number of on-values, and wherein on-values of the plurality of negative control code words are evenly distributed across the N bits such that each ordinal position in the sequence of N bits has a same total number of on-bits from the plurality of negative control code words, and
determining a gene or error associated with the closest matching code word, and
for at least one pixel of the plurality of pixels, storing an association of the pixel with the gene or error.

2. The method of claim 2, wherein N is 16.

3. The method of claim 1, comprising determining a confidence threshold.

4. The method of claim 3, wherein determining the confidence threshold comprises, for each negative control code word, calculating a count of matches of pixels words to the negative control word, thus generating a plurality of counts of matches, and selecting a largest count from the plurality of counts of matches as the confidence threshold.

5. The method of claim 3, comprising, for each gene-identifying code word, calculating a count of matches of pixels words to the gene-identifying code word.

6. The method of claim 5, comprising, for each gene-identifying code word having a count of matches greater than the confidence threshold and each pixel for which the closest matching code word is the gene-identifying code word, storing an association of the pixel with the gene-identifying code word.

7. The method of claim 1, comprising, for each code word of the plurality of code words, calculating a confidence value based on distances between the code word and the pixel words for which the code word is identified as the closest matching code word.

8. The method of claim 1, wherein the plurality of negative control code words comprises between 5% and 25% of the codebook.

9. A spatial transcriptomics system, comprising:
a flow cell to contain a sample to be exposed to fluorescent probes in a reagent;
a plurality of reagent reservoirs, each reagent reservoir including a container to hold a liquid reagent;
a valve system to control flow from one of a plurality of reagent reservoirs to the flow cell;
a pressure source to urge the liquid reagent to flow through the flow cell;
a fluorescence microscope including a variable frequency excitation light source and a camera positioned to receive fluorescently emitted light from the sample; and
a controller configured to
receive a plurality of images of a sample from the fluorescence microscope,
for each pixel of a plurality of pixels registered across the plurality of images, generating a pixel word from intensity values of each pixel of the plurality of pixels of the plurality of images, each pixel word represented by a sequence of N intensity values, and
for each pixel of the plurality of pixels,
compare the pixel word for the pixel to a codebook including a plurality of code words and identifying a closest matching code word of the plurality of code words to the pixel word, each code word represented by a sequence of N bits, wherein the plurality of code words include a plurality of gene-identifying code words and a plurality of negative control code words, wherein the plurality of negative control code words have an equal number of on-values, and wherein on-values of the plurality of negative control code words are evenly distributed across the N bits such that each ordinal position in the sequence of N bits has a same total number of on-bits from the plurality of negative control code words, and
determine a gene or error associated with the closest matching code word, and
for at least one pixel of the plurality of pixels, store an association of the pixel with the gene or error.

10. The system of claim 9, wherein the controller is configured to calculate for each negative control code word a count of matches of pixels words to the negative control word and thus generate a plurality of counts of matches, and to select a largest count from the plurality of counts of matches as a confidence threshold.

11. The system of claim 15, wherein the controller is configured to, for each gene-identifying code word, calculate a count of matches of pixels words to the gene-identifying code word, and for gene-identifying code word having a count of matches greater than the confidence threshold and for each pixel for which the closest matching code word is the gene-identifying code word, store an association of the pixel with the gene-identifying code word.

12. The system of claim 9, wherein the controller is configured to receive the plurality of images of the sample by receiving N images of the sample and each intensity value from the sequence of N intensity values corresponds to one of the N images.

13. The system of claim 9, wherein the plurality of negative control code words comprises between 5% and 25% of the codebook.

14. The system of claim 9, wherein the Hamming distance between any two code words of the plurality of code words is equal.

15. The method of claim 14, wherein the Hamming distance is equal to 4.

16. A computer program product for spatial transcriptomics, the computer program product comprising a non-transitory computer-readable medium having instructions, which, when executed by one or computers, causes the one or computers to:
receive a plurality of images of a sample from an mFISH imaging system;
for each pixel of a plurality of pixels registered across the plurality of images, generate a pixel word from intensity values of each pixel of the plurality of pixels of the plurality of images, each pixel word represented by a sequence of N intensity values; and
for each pixel of the plurality of pixels,
compare the pixel word for the pixel to a codebook including a plurality of code words and identify a closest matching code word of the plurality of code words to the pixel word, each code word represented by a sequence of N bits, wherein the plurality of code words include a plurality of gene-identifying code words and a plurality of negative control code words, wherein the plurality of negative control code words have an equal number of on-values, and wherein on-values of the plurality of negative control code words are evenly distributed across the N bits such that each ordinal position in the sequence of N bits has a same total number of on-bits from the plurality of negative control code words, and
determine a gene or error associated with the closest matching code word, and
for at least one pixel of the plurality of pixels, storing an association of the pixel with the gene or error.

17. The computer program product of claim 16, comprising instructions to determine a confidence threshold wherein for each negative control code word, a count of matches of pixels words to the error correction word is calculated and a plurality of counts of matches is generated to select a largest count from the plurality of counts of matches as the confidence threshold.

18. The computer program product of claim 17, comprising instructions to, for each gene-identifying code word, calculate a count of matches of pixels words to the gene-identifying code word and having a count of matches greater than the confidence threshold and each pixel for which the closest matching code word is the gene-identifying code word, store an associations of the pixel with the gene-identifying code word.

19. The computer program product of claim 16, wherein the instructions to receive the plurality of images of the sample include instructions to receive N images of the sample and each intensity value from the sequence of N intensity values corresponds to one of the N images.

20. The computer program product of claim 16, wherein the plurality of negative control code words comprises between 5% and 25% of the codebook.

21. The computer program product of claim 16, wherein the Hamming distance between any two code words of the plurality of code words is equal.

22. The computer program product of claim 16, wherein the Hamming distance is 4.
